(19) United States
(12) Patent Application Publication
(10) Pub. No.: US 2020/0165218 A1
(43) Pub. Date: May 28, 2020

(54) METHODS FOR MAKING CYCLOHEXENE OXIDE-CONTAINING ESTERS

(71) Applicant: Arkema Inc., King of Prussia, PA (US)

(72) Inventors: Mohammad R. KAZEMIZADEH, Blooming Prairie, MN (US); David E. MAIXNER, Blooming Prairie, MN (US)

(21) Appl. No.: 16/619,587

(22) PCT Filed: Jun. 12, 2018
(86) PCT No.: PCT/US2018/037075

§ 371 Date: Dec. 5, 2019

Related U.S. Application Data
(60) Provisional application No. 62/518,685, filed on Jun. 13, 2017.

(51) Int. Cl. C07D 301/14; C07C 67/08; C07D 303/40; C07D 407/12; C07D 407/14

(52) U.S. Cl. CPC C07D 301/14; C07C 67/08; C07D 407/14; C07D 407/12; C07D 303/40

(57) ABSTRACT

A method of making an ester comprised of at least one cyclohexene oxide moiety is provided, involving the steps of a) esterifying an alcohol with a carboxylic acid-substituted cyclohexene to obtain an intermediate comprised of at least one carboxylate-substituted cyclohexene moiety; and b) epoxidizing the intermediate obtained in step a) with an epoxidizing agent to obtain the ester comprised of at least one cyclohexene oxide moiety. The esters have utility as acid scavengers, plasticizers and reactive resins.

### FIELD OF THE INVENTION

The present invention relates to methods for making cyclohexene oxide-containing esters comprised of one, two, three or more cyclohexane rings having oxirane (epoxide) functional groups, which are useful as, for example, acid scavengers, plasticizers and reactive epoxy resins (e.g., for use in cationic coating applications).

### BACKGROUND OF THE INVENTION

Organic compounds containing one or more cyclohexene oxide rings, such as 7-oxabicyclo (4.1.0) heptane-3-carboxylic acid, 2-ethylhexyl ester, are known to be useful acid scavengers.

7-Oxabicyclo (4.1.0) heptane-3-carboxylic acid, 2-ethylhexyl ester (CAS No. 62256-00-2) has the following structure:

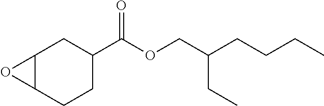 [SMILES: CCCCC(CC)COC(=O)C1CCC2OC2C1]

3-Cyclohexene-1-carboxylic acid, 2-ethylhexyl ester (CAS No. 63302-64-7), the structure of which appears below, is a suitable precursor for the above-mentioned epoxide.

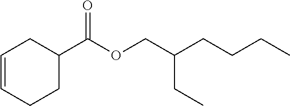 [SMILES: CCCCC(CC)COC(=O)C1CC=CCC1]

The preparation of the above precursor, as well as other cyclohexene carboxylic esters, is described in U.S. Pat. No. 4,076,642. The synthetic route uses the Diels-Alder reaction of butadiene with 2-ethylhexyl acrylates, but has certain disadvantages. Because butadiene is used as a reactant, the reaction must be conducted in a pressurized reactor (e.g., 150 psi at the relatively high reaction temperature of 150° C.). Such conditions raise operational safety concerns and also lead to high costs of production, due to the specialized equipment needed. Moreover, butadiene readily polymerizes under such conditions, leading to lower than desirable yields; such polymerization reactions may be uncontrollable, resulting in possible safety issues. Further, 1,3-butadiene may dimerize to form vinyl cyclohexene, which also has the potential to lower the yield of the desired ester product. The other reactant, ethylhexyl acrylate, will also polymerize in the absence of oxygen; this will also result in lower yields. In addition, the flammability of butadiene requires extensive capital investment if the Diels-Alder reaction is to be made sufficiently safe to be practiced on a commercial scale. The product obtained in such Diels-Alder reaction requires removal of catalyst (aluminum chloride), neutralization, and washing and distillation steps to render it suitable for use in an epoxidation step to obtain the final desired product, 7-oxabicyclo (4.1.0) heptane-3-carboxylic acid, 2-ethylhexyl ester.

Accordingly, there is a need for improved methods of making 3-cyclohexene-1-carboxylic acid, 2-ethylhexyl ester and other such related precursor compounds that avoid or minimize the above-mentioned problems associated with the known synthetic route involving the use of butadiene in a Diels-Alder reaction. At the same time, it would also be desirable to develop methods for making other types of cyclohexene oxide-containing esters that are not readily accessible synthetically using Diels-Alder chemistry.

### SUMMARY OF THE INVENTION

The present invention, according to certain aspects, provides a synthetic method for the preparation of cyclohexene oxide-containing esters that avoids some or all of the above-mentioned disadvantages associated with the conventional Diels-Alder route. In particular, the inventive method does not utilize a volatile diene that is susceptible to polymerization and dimerization and that requires relatively expensive processing equipment. Moreover, the method, unlike the conventional Diels-Alder reaction scheme, does not require filtration or distillation and can be performed in a simple (low cost) reactor equipped with heating and mixing means, at lower temperatures and at atmospheric pressure. The esterification product obtained from the initial esterification step may utilize an acidic catalyst, which (unlike the catalyst used to carry out a Diels-Alder reaction in the conventional process) does not need to be removed prior to subjecting the esterification product to a further epoxidation step. In fact, the acidic catalyst employed for the esterification (e.g., methane sulfonic acid) can be left in the esterification product and used as a catalyst during the epoxidation step as well. The present inventive method is capable of directly producing a cyclohexene oxide-containing ester in high yield and high purity (e.g., at least 97% purity), thereby avoiding the need to perform multiple or complicated purification and isolation steps (thereby lowering production costs).

As will be explained in more detail subsequently, the present invention provides a method of making an ester comprised of at least one cyclohexene oxide moiety, comprising:
a) esterifying an alcohol with a carboxylic acid-substituted cyclohexene to obtain an intermediate comprised of at least one carboxylate-substituted cyclohexene moiety (sometimes hereinafter referred to as “the esterification step”); and
b) epoxidizing the intermediate obtained in step a) with an epoxidizing agent to obtain the ester comprised of at least one cyclohexene oxide moiety (sometimes hereinafter referred to as “the epoxidation step”).

By practicing the above-mentioned method, compounds useful as acid scavengers, plasticizers, and reactive resins (in coating compositions, for example) can be prepared, including compounds having the following structures (I) and (II):

Q-C(═O)O—R (I)
wherein Q is a cyclohexene oxide moiety and R is a C3-C22 alkyl group functionalized with one or more epoxide groups;

A-(OC(═O)Q)ₓ (II)
wherein A is an organic moiety, Q is a cyclohexene oxide moiety and x is an integer of 2 or more (e.g., 2-6).

### DETAILED DESCRIPTION OF THE INVENTION

### Starting Materials

The organic compounds utilized as starting materials in the present invention include alcohols and carboxylic acid-substituted cyclohexenes, wherein in one step an intermediate comprised of at least one carboxylate-substituted cyclohexene moiety is obtained by esterifying an alcohol with a carboxylic acid-substituted cyclohexene.

The types of alcohols suitable for use are not particularly limited and may be any organic compound containing one or more hydroxyl (—OH) groups per molecule. For example, the alcohol may be a monoalcohol (containing a single hydroxyl group per molecule) or a polyalcohol (polyol) containing two, three, four, five or more hydroxyl groups per molecule. The hydroxyl group may be a primary, secondary or tertiary hydroxyl group, with primary and secondary hydroxyl groups generally being preferred; when the alcohol is a polyol, the alcohol may contain a single type of hydroxyl group (e.g., each of the hydroxyl groups may be a primary hydroxyl group) or a combination of different types of hydroxyl groups (e.g., at least one primary hydroxyl group and at least one secondary hydroxyl group).

The alcohol may be an aliphatic alcohol. The aliphatic alcohol may be a saturated aliphatic alcohol or an unsaturated aliphatic alcohol (containing, for example, one or more carbon-carbon double bonds or sites of ethylenic unsaturation). The carbon-carbon double bonds may be in terminal (alpha) positions (providing the structure —CR═CH₂, for example, where R is H or an organic group such as an alkyl group) and/or appear at internal positions along a chain (providing, for example, the structure —CR═CR′R″ where R is H or an organic group such as an alkyl group, R′ is H or an organic group such as an alkyl group and R″ is an organic group such as an alkyl group). Such sites of ethylenic unsaturation may be capable of being epoxidized in the epoxidation step of the present invention, thereby providing one or more epoxide functional groups in addition to the epoxide functional group(s) present in the cyclohexane moiety or moieties of the product obtained from the epoxidation step. Suitable aliphatic alcohols include straight chain (linear), branched and cyclic aliphatic alcohols, both saturated and unsaturated. As used herein, the term “aliphatic alcohols” also includes alcohols which are aliphatic and which contain one or more oxygen atoms along a hydrocarbon chain (forming ether groups).

Examples of suitable saturated monoalcohols include, but are not limited to, C₁ to C₂₂ saturated monoalcohols such as methanol, ethanol, n-propanol, isopropanol, n-butanol, sec-butyl alcohol, n-pentanol, n-hexanol, n-heptanol, n-octanol, 3-methyl-3-pentanol, pelargonic alcohol, 1-decanol, saturated fatty alcohols (e.g., lauryl alcohol, stearyl alcohol, undecyl alcohol, tridecyl alcohol, myristyl alcohol, pentadecyl alcohol, cetyl alcohol, nonadecyl alcohol, 1-eicosanol, 1-heneicosanol), cyclohexanol, cyclohexane ethanol, cyclohexane methanol, 4-methylcyclohexane methanol, menthol, 2-ethoxyethanol, isobornyl alcohol, 2-methoxyethanol, 2-methyl-2-butanol, 3-methylbutanol, 2-methyl-1-propanol, and the like.

Alkoxylated monoalcohols, such as monoalcohols which have been reacted with one or more moles of an alkylene oxide such as ethylene oxide and/or propylene oxide per mole of monoalcohol are also suitable for use as the alcohol starting material in the present invention.

Examples of suitable unsaturated monoalcohols include C3 to C22 monoalcohols containing one, two, three, four or more sites of ethylenic unsaturation, such as, for example, allyl alcohol, cis-3-hexen-1-ol, 4-penten-1-ol, cis-3-penten-1-ol, 3-buten-1-ol, trans-2-pentene, 5-hexen-1-ol, 2-ethylbut-2-en-1-ol, unsaturated fatty alcohols (e.g., palmitoleyl alcohol, oleyl alcohol, linoleyl alcohol, erucyl alcohol), 1-cyclohexene-1-ethanol, 2-cyclohexene-1-ethanol, 3-cyclohexene-1-ethanol, 1-cyclohexene-1-methanol, 2-cyclohexene-1-methanol, 3-cyclohexene-1-methanol, 4-penten-2-ol, 5-hexen-2-ol, 6-methyl-5-hepten-2-ol, carveol, alpha-terpineol, linalool, citronellol, dicyclopentadiene alcohol and the like.

Examples of suitable polyalcohols (polyols) include, but are not limited to, C2 to C22 aliphatic diols such as ethylene glycol, 1,2-propylene glycol, 1,3-propylene glycol, 1,2-, 1,3- and 1,4-butanediol 1,2-, 1,3-, 1,4- and 1,5-pentanediol, 1,2-, 1,3-, 1,4-, 1,5-, and 1,6-hexanediol, 2-methyl-1,3-propanediol, neopentyl glycol, glycerol, sugars (e.g., mono- and di-saccharides, such as sucrose), sugar alcohols (e.g., sorbitol), pentaerythritol, dipentaerythritol, tripentaerythritol, trimethylolpropane, trimethylolethane, 3-methyl-1,5-pentanediol, 1,4-cyclohexanedimethanol, 1,3-cyclohexanedimethanol, and the like. Alkoxylated polyalcohols, such as polyalcohols which have been reacted with one or more moles of an alkylene oxide such as ethylene oxide and/or propylene oxide per mole of polyalcohol are also suitable for use as the alcohol starting material in the present invention. Oligomers of ethylene glycol, propylene glycol, butylene glycol and the like may also be used as polyalcohol starting materials in the present invention. Suitable polyalcohols also include bisphenols such as bisphenol A and alkoxylated derivatives thereof.

Other examples of suitable alcohols include vegetable based polyols that are the product of epoxy ring-opening reactions using either aqueous acid or base with epoxidized fatty acid esters derived from epoxidized algae oil, epoxidized canola oil, epoxidized coconut oil, epoxidized castor oil, epoxidized corn oil, epoxidized cottonseed oil, epoxidized flax oil, epoxidized fish oil, epoxidized grapeseed oil, epoxidized hemp oil, epoxidized jatropha oil, epoxidized jojoba oil, epoxidized mustard oil, epoxidized canola oil, epoxidized palm oil, epoxidized palm stearin, epoxidized rapeseed oil, epoxidized safflower oil, epoxidized soybean oil, epoxidized sunflower oil, epoxidized tall oil, epoxidized olive oil, epoxidized tallow, epoxidized lard, epoxidized chicken fat, epoxidized linseed oil, epoxidized tung oil, epoxidized linseed oil, epoxidized tung oil and mixtures thereof. It is to be understood that complete epoxidation of these compounds is not necessary in the practice of the invention, nor is complete ring-opening of the epoxidized compound(s). Hydroxyl values ranging from 1-400 are suitable.

Aromatic alcohols, i.e., alcohols containing one or more aromatic rings per molecule with at least one hydroxyl group bonded indirectly to an aromatic ring, may also be utilized in the present invention. Examples of suitable aromatic alcohols include benzyl alcohol, phenethyl alcohol, indanol, 1-phenyl-1-propanol, 2-phenyl-1-propanol, 1,2,3,4-tetrahydro-1-naphthol and the like, including alkoxylated and substituted derivatives thereof. Phenols constitute another type of alcohol that can be employed as a starting material.

The alcohol may be substituted with one or more substituents, provided that such substituent(s) does or do not interfere with the desired esterification and epoxidation steps of the present invention. Such substituents, which may be considered to take the place of hydrogen atoms, may include, for example, halogens (F, Cl, Br, I), cyano groups, nitro groups, alkoxy groups and the like.

In one embodiment of the invention, the alcohol may be a monoalcohol corresponding to the formula ROH, wherein R is a saturated linear, branched or cyclic alkyl group containing from 1 to 22 carbon atoms, an unsaturated linear, branched or cyclic alkylene group containing from 3 to 22 carbon atoms and from 1 to 6 carbon-carbon double bonds (sites of ethylenic unsaturation), or an aralkyl group (such as benzyl or phenethyl) containing from 8 to 22 carbon atoms.

In another embodiment, the alcohol may be a polyalcohol (polyol) corresponding to the formula HOCH₂(CR¹R²)ₙCH₂OH, wherein n is 0 or an integer of from 1 to 20, R¹ and R² are independently selected from H, alkyl (in particular, a C₁ to C₁₂ alkyl group), hydroxyl (—OH), aryl, or hydroxyalkyl (in particular, a C₁ to C₁₂ hydroxyalkyl group, such as —CH₂OH, —CH₂CH₂OH or —CH₂CH₂CH₂OH), subject to the proviso that R¹ and R² attached to the same carbon atom are not both hydroxyl and to the understanding that when n is 2 or more the R¹ groups may be the same as or different from each other and the R² groups may be the same as or different from each other.

Carboxylic acid-substituted cyclohexenes suitable for use in the present invention include organic compounds characterized by containing at least one cyclohexene ring which is substituted by a carboxylic acid group (—CO₂H). The cyclohexene ring(s) may be substituted by one or more substituents other than carboxylic acid groups, such as alkyl (e.g., methyl, ethyl), aryl (e.g., phenyl), halogen, cyano, alkoxy, nitro and the like, provided that such substituents do not interfere with the ability to carry out the desired esterification and epoxidation reactions. The cyclohexene ring may contain a single carbon-carbon double bond (i.e., a single site of ethylenic unsaturation), which may appear at the 1, 2 or 3 position of the cyclohexene ring. Methods of making carboxylic acid-substituted cyclohexenes are well known in the art and are described, for example, in U.S. Pat. Nos. 2,653,167 and 3,305,579. 3-Cyclohexene-1-carboxylic acid (sometimes also referred to herein as “3-CHA”) is a particularly preferred carboxylic acid-substituted cyclohexene which is readily available from commercial sources. Other exemplary carboxylic acid-substituted cyclohexenes suitable for use in the present invention include, but are not limited to, 1-cyclohexene-1-carboxylic acid, 2-cyclohexene-1-carboxylic acid, 4-methyl-3-cyclohexene-1-carboxylic acid and 2-phenyl cyclohexene-3-carboxylic acid.

### Esterification

In the esterification step of the present invention, an alcohol is contacted with a carboxylic acid-substituted cyclohexene under conditions effective to achieve at least partial esterification of the hydroxyl group(s) of the alcohol by the carboxylic acid-substituted cyclohexene. In such an esterification reaction, a hydroxyl group of the alcohol is converted to an ester group (—OH→—OC(═O)R, wherein the —C(═O)R moiety is contributed by the carboxylic acid-substituted cyclohexene). As a result of the esterification, water is generated. To assist in driving the esterification to completion, it will generally be desirable to remove the water so formed from the reaction mixture, such as by distillation (including under vacuum), sparging, use of dehydrating agents or azeotropic agents, or other techniques known in the field of organic chemistry.

Typically, it will be desirable to employ an amount of carboxylic acid-substituted cyclohexene that is approximately stoichiometric with respect to the amount of alcohol, although it may be advantageous to have one of the starting materials (in particular, the alcohol) present in moderate excess relative to the amount of the other starting material. For example, the molar ratio of hydroxyl groups (contributed by the alcohol) to carboxylic acid groups (contributed by the carboxylic acid-substituted cyclohexene) may be, in various embodiments of the invention, from 1:1.5 to 1.5:1, or from 1:1.4 to 1.4:1, or from 1:1.3 to 1.3:1, or from 1:1.2 to 1.2:1, or from 1:1.1 to 1.1:1, or approximately 1:1.

A catalyst may be present in the esterification reaction mixture for the purpose of accelerating the rate of reaction between the starting materials. Acid catalysts are particularly preferred for this purpose. A relatively strong acid catalyst may be used; for example, the acid catalyst may have a pKa of less than −1.74. Examples of suitable acid catalysts include, but are not limited to, sulfuric acid and sulfonic acids such as methane sulfonic acid. Generally speaking, the amount of catalyst present is relatively low, e.g., not more than about 0.5% by weight based on the total weight of the starting materials. The catalyst may be homogeneous (soluble in the esterification reaction mixture) or heterogeneous (insoluble in the esterification reaction mixture).

The esterification temperature may vary depending upon the reactivity of the starting materials and the type of catalyst present (if any), among other factors. Generally speaking, such temperature will be selected to provide a suitably fast rate of reaction, while avoiding or minimizing the amount of any undesired by-products which may be generated during the esterification. For example, where a strong acid catalyst is employed as previously described, reaction temperatures of from about 75° C. to about 160° C. will typically be suitable. Removing the water of reaction from the reaction mixture as the esterification reaction progresses may permit the reaction to be conducted at a lower temperature, whilst still achieving the desired extent of conversion of the starting materials within a predetermined period of time.

The esterification reaction is carried out for a time and at a temperature effective to reach the desired percent conversion of the starting materials and the desired yield of the esterified intermediate. Generally speaking, reaction times of from about 2 to about 8 hours will be suitable, although shorter or longer reaction times may be appropriate or desired depending upon the starting materials, type of catalyst used (if any) and other conditions. Typically, the reaction conditions are selected so that at least 50%, at least 60%, at least 70%, at least 80%, at least 90% or at least 95% conversion of one or both of the starting materials is achieved.

The esterification reaction may be carried out in the absence of any solvent. However, in other embodiments, one or more solvents, in particular one or more organic solvents which are non-reactive (i.e., containing neither hydroxyl groups or carboxylic acid groups), may be utilized.

In one embodiment of the invention, wherein an aliphatic alcohol containing primary and/or secondary hydroxyl groups and 3-cyclohexene-1-carboxylic acid are used as starting materials in a neat reaction (i.e., no solvent is present), with a strong acid such as sulfuric acid or a sulfonic acid present as an esterification catalyst, the starting materials are reacted at a temperature of from about 110° C. to about 150° C. under a partial vacuum (e.g., about 10 to about 30 inches Hg) to remove the water formed during the esterification, with such reaction being carried out for a time effective to remove at least 80% or at least 90% of the theoretical amount of the water of reaction.

Following the esterification reaction, the product obtained may be subjected to one or more processing and/or purification steps prior to carrying out epoxidation of the desired intermediate ester. For example, techniques such as distillation, stripping (to remove any unreacted starting material), neutralization, fractionation, or the like may be employed.

However, in other embodiments of the invention, the esterification reaction product is carried on directly to the epoxidation step, without any further processing or purification being performed. In particular, any residual acid in the esterification reaction product may be left in so that it is present during the epoxidation step as well.

### Epoxidation

In the epoxidation step of the present invention, one or more of the ethylenically unsaturated sites present in the intermediate esterification product (the intermediate comprised of at least one carboxylate-substituted cyclohexene moiety) is epoxidized using a suitable epoxidizing agent or combination of epoxidizing agents. The epoxidation step thus introduces one or more epoxide functional groups into the intermediate esterification product, yielding the desired ester comprised of at least one cyclohexene oxide moiety. In particular, a carbon-carbon double bond present in a cyclohexene moiety of the intermediate esterification product is epoxidized and converted into an epoxy group. Where the intermediate esterification product contains two or more cyclohexene moieties per molecule, at least one of the cyclohexene moieties is so converted. In one embodiment of the invention, all such cyclohexene moieties are epoxidized. If the intermediate esterification product contains one or more sites of ethylenic unsaturation other than as part of a cyclohexene moiety, one or more such ethylenically unsaturated sites may also be epoxidized. Thus, by utilizing the process of the present invention, the epoxidation product may be a compound containing one, two, three, four or more epoxide groups.

Any of the epoxidizing agents known in the art to be capable of oxidizing a carbon-carbon double bond so as to introduce a three membered ring containing oxygen and the two carbon atoms originating from the carbon-carbon double bond may be utilized in the present invention. The epoxidizing agent may be an organic or inorganic epoxidizing agent, for example. The epoxidation may be carried out in the presence of a suitable catalyst (e.g. a metal-containing epoxidation catalyst), in addition to the epoxidizing agent. In an especially preferred embodiment of the invention, the epoxidizing agent may be a peroxy compound, i.e., a compound containing at least one —O—O— functional group. Suitable peroxy compounds include, for example, hydrogen peroxide, hydroperoxides, peroxides, peresters, and peracids and combinations thereof. The peroxy compound(s) may be formed in situ; for example, a peracid may be produced in situ using hydrogen peroxide and a carboxylic acid such as formic acid or acetic acid as starting materials. Peracetic acid is an example of an epoxidizing agent that is suitable for use in the present application. Organic hydroperoxides, such as tert-butyl hydroperoxide, ethylbenzene hydroperoxide or cumene hydroperoxide, constitute another suitable type of epoxidizing agent. Molybdenum complexes may be employed to catalyze epoxidation by an organic hydroperoxide. Enzymatic and chemo-enzymatic epoxidation may also be utilized.

The stoichiometry of the esterification product and epoxidizing agent may be varied as may be desired in order to achieve the desired yield and selectivity of the epoxidation product which is the intended target. Generally speaking, it will be desirable for the number of moles of peroxy groups supplied by the epoxidizing agent to be approximately equal to or somewhat greater than the number of moles of carbon-carbon double bonds present in the esterification product to be epoxidized. For example, the molar ratio of double bonds (C═C) to peroxy (—OO—) may be from about 1:1 to about 1:1.4, or from about 1:1.1 to about 1:1.3 or about 1:1.2.

The epoxidizing agent and esterification product are contacted for a time and at a temperature effective to achieve the desired degree of conversion of the carbon-carbon double bonds in the esterification product to epoxy (oxirane) groups. The extent of epoxidation may, for example, be monitored by gas chromatography (e.g., by comparing the relative peak areas of the peaks associated with the intermediate esterification product and with the desired epoxidation product) or by measuring the iodine value of the epoxidation reaction product.

The epoxidation may be carried out in the liquid phase, either neat or in the presence of a suitable solvent or mixture of solvents (which may be water and/or one or more organic solvents). A two phase epoxidation system may be employed, for example. The pH of the reaction medium may be adjusted as may be desired, using a suitable acid, base and/or buffer system. In one embodiment of the invention, the epoxidizing agent(s) may be added to the intermediate esterification product, either continuously or portion-wise. A catalyst may be present in the reaction mixture to facilitate or accelerate the desired epoxidation of carbon-carbon double bonds.

According to various advantageous embodiments, epoxidation is carried out under conditions effective to achieve at least 80%, at least 85%, at least 90%, at least 95% or even at least 99% conversion of the ethylenically unsaturation present in the intermediate esterification product to epoxy functionality.

When a percarboxylic acid such as peracetic acid is employed as an epoxidizing agent, it may be advantageous to carry out the epoxidation in stages in order to avoid or reduce the issues sometimes encountered (e.g., ring-opening of the desired epoxide product) when the concentration of the co-product carboxylic acid in the reaction mixture becomes too great. For example, a first (less than stoichiometric) amount of percarboxylic acid may be combined and reacted with the esterification product, then the intermediate reaction product purified to remove at least some of the carboxylic acid co-product before reacting the intermediate reaction product with one or more further portions of the percarboxylic acid.

Following epoxidation, the reaction product may be subjected to one or more further processing or purification steps in order to recover the desired ester comprised of at least one cyclohexene oxide moiety in a purity appropriate for its intended further use. Such further steps may include, for example, washing (e.g., with water), neutralization (e.g., by washing with water containing a base), phase separation, distillation, and/or drying and the like.

### Epoxidation Products

The above-described synthetic procedures may be employed to produce a wide variety of cyclohexene oxide moiety-containing esters, the structures of which may be varied as may be desired by selection of particular combinations of the alcohol and carboxylic acid-substituted cyclohexene used as starting materials in step a). The reaction products may, for example, be esters containing a single epoxy group, such as 7-oxabicyclo (4.1.0) heptane-3-carboxylic acid, 2-ethylhexyl ester (a known compound). However, it is also possible to produce esters containing two or more epoxy groups per molecule, at least some of which may be previously unknown compounds. For example, esters containing a plurality of epoxy groups may be obtained by employing an unsaturated alcohol as a starting material, wherein the alcohol contains one, two, three or more carbon-carbon double bonds that are oxidized during the epoxidation step to provide epoxy groups. Such esters may correspond to structure (I):

Q-C(═O)O—R (I)
wherein Q is a cyclohexene oxide moiety (e.g., a 3-cyclohexene oxide moiety) and R is a C3-C22 alkyl group functionalized with one or more epoxide groups (derived from the starting unsaturated alcohol).

Another way to achieve esters containing two or more epoxy groups per molecule is to utilize a polyol as the alcohol starting material. Two or more of the hydroxyl groups of the polyol are esterified with a carboxylic acid-substituted cyclohexene during the esterification step, thereby incorporating two or more cyclohexene rings in the intermediate esterification product. These cyclohexene rings then undergo epoxidation in the epoxidation step, thereby yielding a plurality of cyclohexene oxide rings. Such reaction products may correspond to structure (II):

A-(OC(═O)Q)ₓ (II)
wherein A is an organic moiety (derived from the starting polyol), Q is a cyclohexene oxide moiety (e.g., a 3-cyclohexene oxide moiety) and x is an integer of 2 or more. For example, where the starting polyol is pentaerythritol, A is —CH₂CR′₂CH₂—, with R′=CH₂—, and x=4.

### End Uses for Epoxidation Products

The cyclohexene oxide-containing esters which are the subject of the present invention are useful in a wide variety of applications. For example, they may be used as acid scavengers and corrosion inhibitors (to scavenge acid in aviation hydraulic fluids or other lubricant or functional fluid compositions), as plasticizers (to plasticize polymer compositions), or as the primary resin in formulations for cationic coatings (wherein a coating composition is cured by a cationic curing process to provide a coating on a surface of a substrate). They are also useful as synthetic intermediates in the preparation of other compounds, such as fragrances and adhesives.

Various exemplary and non-limiting aspects of the present invention may be summarized as follows.

Aspect 1: A method of making an ester comprised of at least one cyclohexene oxide moiety, comprising:
a) esterifying an alcohol with a carboxylic acid-substituted cyclohexene to obtain an intermediate comprised of at least one carboxylate-substituted cyclohexene moiety; and
b) epoxidizing the intermediate obtained in step a) with an epoxidizing agent to obtain the ester comprised of at least one cyclohexene oxide moiety.

Aspect 2: The method of Aspect 1, wherein the alcohol is a mono-alcohol.

Aspect 3: The method of Aspect 1 or 2, wherein the alcohol is a C1 to C24 linear or branched aliphatic mono-alcohol or cycloaliphatic mono-alcohol.

Aspect 4: The method of Aspect 1, wherein the alcohol is a polyol.

Aspect 5: The method of Aspect 1 or 4, wherein the alcohol is a polyol selected from the group consisting of ethylene glycol; 1,2-propylene glycol; 1,3-propylene glycol; 1,2-, 1,3- and 1,4-butanediol; 1,2-, 1,3-, 1,4- and 1,5-pentanediol; 1,2-, 1,3-, 1,4-, 1,5-, and 1,6-hexanediol; 2-methyl-1,3-propanediol; neopentyl glycol; glycerol; sugars; sugar alcohols, pentaerythritol; dipentaerythritol; tripentaerythritol; trimethylolpropane; trimethylolethane; 3-methyl-1,5-pentanediol; 1,4-cyclohexanedimethanol; 1,3-cyclohexanedimethanol; oligomers of ethylene glycol, propylene glycol, and butylene glycol; bisphenols; and alkoxylated derivatives thereof

Aspect 6: The method of any of Aspects 1 to 4, wherein the alcohol contains at least one carbon-carbon double bond.

Aspect 7: The method of Aspect 1, wherein the alcohol is selected from the group consisting of 2-ethyl hexyl alcohol, ethylene glycol, 1,2-propylene glycol, pentaerythritol, oleyl alcohol, undecelynic alcohol, benzyl alcohol, and dodecyl alcohol.

Aspect 8: The method of any of Aspects 1 to 7, wherein the epoxidizing agent is a peroxy compound.

Aspect 9: The method of any of Aspects 1 to 8, wherein the epoxidizing agent is a percarboxylic acid.

Aspect 10: The method of any of Aspects 1 to 9, wherein the percarboxylic acid is peracetic acid.

Aspect 11: The method of any of Aspects 1 to 10, wherein step a) is carried out in the presence of an acid catalyst.

Aspect 12: The method of Aspect 11, wherein the acid catalyst has a pKa of less than −1.74.

Aspect 13: The method of Aspect 11, wherein the acid catalyst is selected from the group consisting of sulfonic acids and sulfuric acid.

Aspect 14: The method of any of Aspects 1 to 13, wherein water of reaction is removed during step a).

Aspect 15: The method of any of Aspects 1 to 14, wherein the carboxylic acid-substituted cyclohexene is 3-cyclohexene-1-carboxylic acid.

Aspect 16: A compound having structure (I):

Q-C(═O)O—R (I)
wherein Q is a cyclohexene oxide moiety and R is a C3-C22 alkyl or alicyclic group functionalized with one or more epoxide groups.

Aspect 17: A compound having structure (II):

A-(OC(═O)Q)ₓ (II)
wherein A is an organic moiety, Q is a cyclohexene oxide moiety and x is an integer of 2 or more.

Within this specification, embodiments have been described in a way which enables a clear and concise specification to be written, but it is intended and will be appreciated that embodiments may be variously combined or separated without departing from the invention. For example, it will be appreciated that all preferred features described herein are applicable to all aspects of the invention described herein.

In some embodiments, the invention herein can be construed as excluding any element or process step that does not materially affect the basic and novel characteristics of the methods described herein. Additionally, in some embodiments, the invention can be construed as excluding any element or process step not specified herein.

Although the invention is illustrated and described herein with reference to specific embodiments, the invention is not intended to be limited to the details shown. Rather, various modifications may be made in the details within the scope and range of equivalents of the claims and without departing from the invention.

### EXAMPLES

### Example 1

The following process is used to prepare 7-oxabicyclo (4.1.0) heptane-3-carboxylic acid, 2-ethylhexyl ester (CAS No. 62256-00-2). Two commercially available products (3-cyclohexene-1-carboxylic acid (3-CHA) and 2-ethylhexyl alcohol (2-EH) were used as starting materials to prepare 3-cyclohexene-1-carboxylic acid 2-ethylhexyl ester, the olefin employed to make the desired epoxide.

In a 250 mL flask equipped with an agitator and Dean Stark trap, charge 100 grams 3-CHA, 124 grams 2-EH (20% excess) and 0.20 grams 70% methane sulfonic acid. The mixture is heated under partial vacuum (20 inches Hg) at 130° C. After collecting 14 mL water, the excess alcohol and 3-CHA are stripped off under vacuum at 110° C. The resulting product is then epoxidized using buffered peracetic acid with a pH of 4, as described below.

180 grams of 3-cyclohexene-1-carboxylic acid 2-ethylhexyl ester obtained from the above-described reaction is charged into a 500 mL three neck flask. Then, 55 grams of 20% peracetic acid, which is adjusted with caustic to a pH of 4, is added over one hour. The mixture is mixed at 40° C. for another hour and then the aqueous phase is separated. The same step is repeated six to seven more times. The progress of the peroxidation reaction is monitored with GC (gas chromatography) until the GC peak related to 3-cyclohexene-1-carboxylic acid 2-ethylhexyl ester is less than 3%. At this point, after removing the aqueous phase, the oil layer is washed with water and 5% sodium bicarbonate to remove any residue of acid, then the oil layer is dried under vacuum at 100° C. The epoxide product from this process had the following properties:

| | |
|---|---|
| Oxirane | 5.93% |
| A.V. | .23 |
| Color (APHA) | 92 |
| Oxide by GC | 96.5% |

The above reaction sequence may be illustrated as follows:

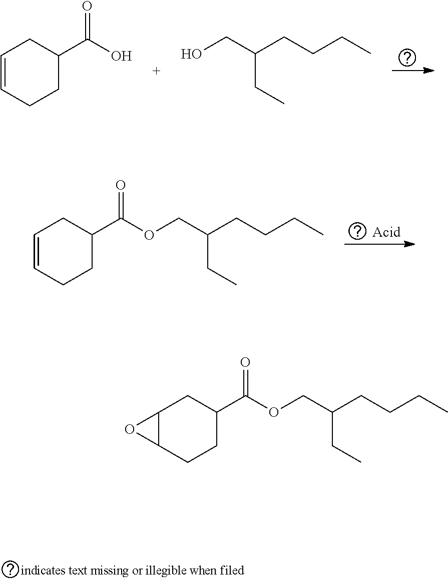

### Example 2

Example 2 is similar to Example 1, except that sulfuric acid (H₂SO₄) is used as an esterification catalyst.

In a 250 ml flask equipped with an agitator and a Dean Stark trap, charge 100 grams 3-cyclohexene-1 carboxylic acid and 124 grams 2 ethyl hexyl alcohol (20% excess) and 0.20 grams 93% sulfuric acid. The mixture is heated under partial vacuum (20 inches Hg) at 130° C. After collecting 14 milliliters of water, the excess alcohol and 3-CHA are stripped off under vacuum at 110° C. Then, the product is epoxidized using buffered peracetic acid with a pH of 4 as described below.

180 grams of 3-cyclohexene-1-carboxylic acid 2-ethylhexyl ester from the above reaction is charged into a 500 ml three neck flask. Then, over the course of an hour, 55 grams of 20% peracetic acid, which has been adjusted with caustic to pH 4, is added. The mixture is mixed at 40° C. for another hour and then the aqueous phase is separated. The same step is repeated six to seven more times. The epoxidation progress is monitored with GC (gas chromatography) until the GC peak related to 3-cyclohexene-1-carboxylic acid 2-ethylhexyl ester is less than 3%. At this point, after removing aqueous phase, the oil layer is washed with water followed by 5% sodium bicarbonate and another water wash to remove any residue of acid. Then the oil layer dried under vacuum at 100° C. The epoxide product from this process has the following properties:

| | |
|---|---|
| Oxirane | 5.9% |
| A.V. | .26 |
| Color (APHA) | 96 |
| Oxide by GC | 97.4% |

### Example 3

This example demonstrates the preparation of ethylene glycol dicyclohexane carboxylate epoxide (containing two cyclohexene oxide moieties per molecule) by reacting 3-cyclohexane carboxylic acid (3-CHA) with ethylene glycol and then epoxidizing the resulting ester: The following illustrates the reaction sequence:

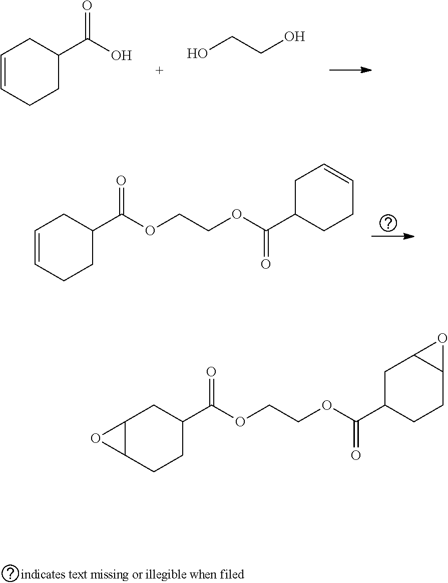

In a 500 ml three neck flask equipped with an agitator and a Dean Stark trap, charge 215 grams 3-cyclohexene-1-carboxylic acid, 48 grams ethylene glycol and 0.40 grams 70% methane sulfonic acid. The mixture is heated under partial vacuum (20 inches Hg) at 130° C., after collecting 68.5 mL water (theoretical 71.5 mL). The excess of 3-CHA is stripped off under vacuum at 110° C. The ester product thus obtained has the following analysis:

| | |
|---|---|
| 3-CHA | 0.4% |
| Mono ester | 0.3% |
| Di ester | 96.2% |
| Iodine value | 180 (theoretical = 182) |

The ester product is then epoxidized using buffered peracetic acid with a pH of 4 as described below.

180 grams of ethylene glycol dicyclohexene carboxylate from above-described esterification reaction is charged into a 500 ml three neck flask and then 45 grams of 20% peracetic acid, which had been adjusted with caustic to pH 5, are added over one hour. The mixture is mixed at 40° C. for another hour and then the aqueous phase is separated. The same step was repeated eight to ten more times. The epoxidation progress is monitored with GC until the GC peak related to ethylene glycol di-cyclohexene carboxylate ester disappears and the content of mono epoxy ethylene glycol dicyclohexane carboxylate ester was less than 3%. At this point of reaction, after removing the aqueous phase, the oil layer is washed with water and 5% sodium bicarbonate to remove any residue of acid, then the oil layer is dried under vacuum at 100° C.

### Example 4

A diepoxide product is prepared by esterifying 1,2-propylene glycol with 3-CHA and then epoxidizing the esterification product, in accordance with the following general reaction scheme.

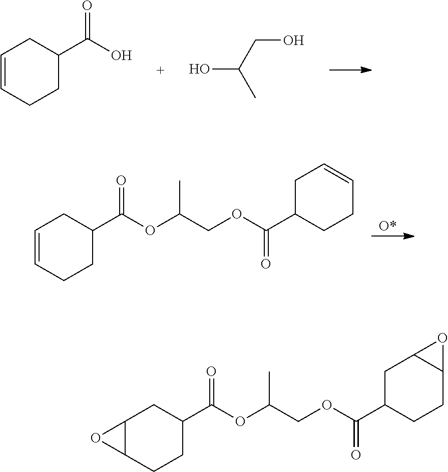

### Example 5

A tetraepoxide product is prepared by esterifying pentaerythritol with 3-CHA and then epoxidizing the esterification product, in accordance with the following general reaction scheme.

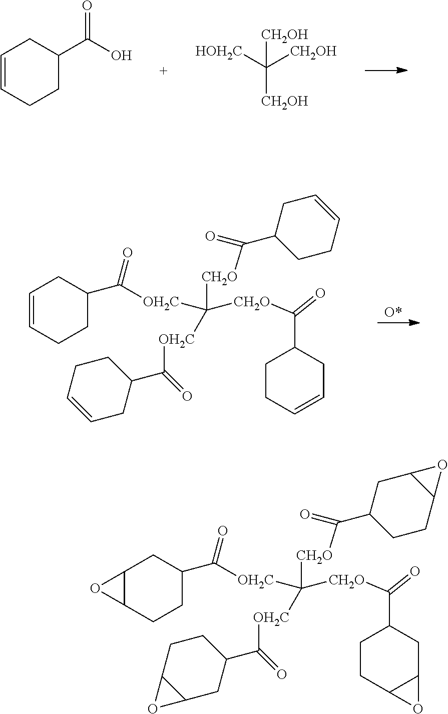

### Example 6

A diepoxide product, wherein one epoxy functional group is on a cyclohexane ring and the other epoxy group is present in a long chain linear alkyl group, is prepared by esterifying oleyl alcohol with 3-CHA and then epoxidizing the esterification product as illustrated in the following reaction scheme.

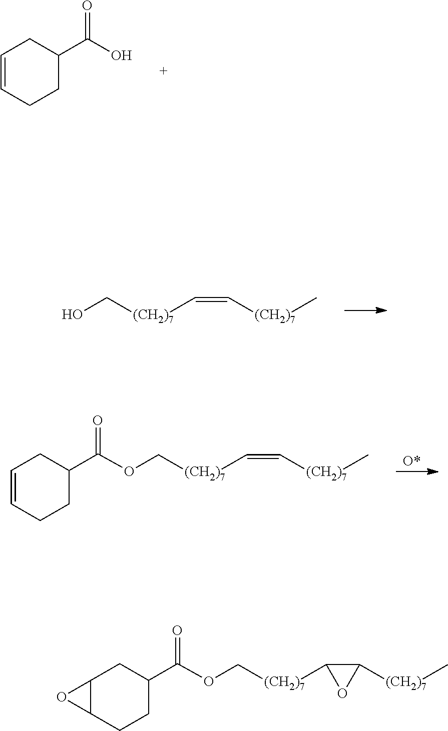

### Example 7

A diepoxide product, wherein one epoxy functional group is on a cyclohexane ring and the other epoxy group is present in the alpha (terminal) position of a long chain linear alkyl group, is prepared by esterifying undecelynic alcohol with 3-CHA and then epoxidizing the esterification product as illustrated in the following reaction scheme.

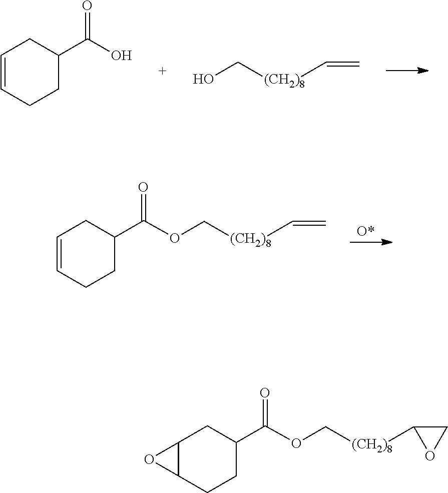

### Example 8

A monoepoxide expected to have utility as an acid scavenger is prepared by reacting 3-CHA with benzyl alcohol to form an ester product which is subsequently epoxidized, in accordance with the following reaction scheme.

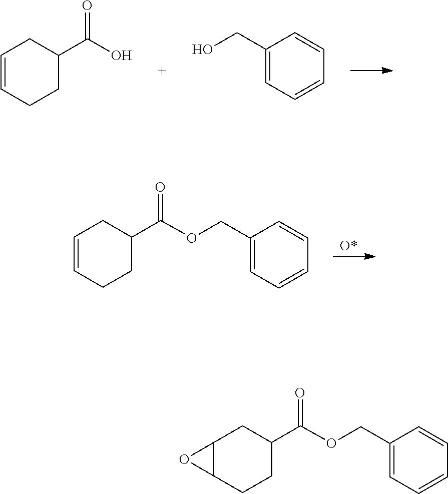 [SMILES: O=C(O)C1CC=CCC1 ; OCc1ccccc1 ; O=C(OCc1ccccc1)C1CC=CCC1 ; O=C(OCc1ccccc1)C1CCC2OC2C1]

### Example 9

A monoepoxide expected to have utility as an acid scavenger is prepared by reacting 3-CHA with dodecyl alcohol to form an ester product which is subsequently epoxidized, in accordance with the following reaction scheme.

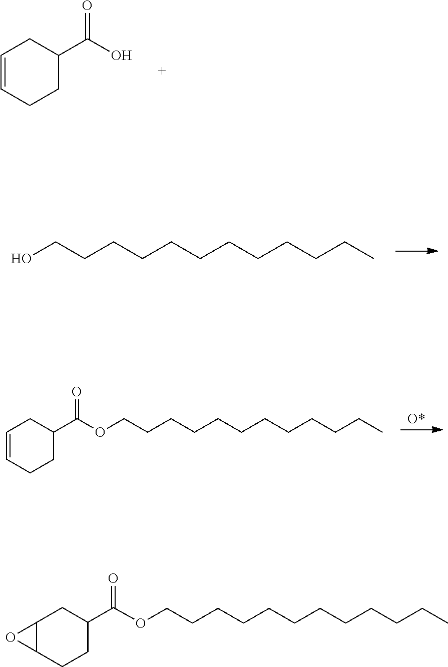 [SMILES: O=C(O)C1CC=CCC1 ; CCCCCCCCCCCCO ; CCCCCCCCCCCCOC(=O)C1CC=CCC1 ; CCCCCCCCCCCCOC(=O)C1CCC2OC2C1]

1. A method of making an ester comprised of at least one cyclohexene oxide moiety, comprising:
a) esterifying an alcohol with a carboxylic acid-substituted cyclohexene to obtain an intermediate comprised of at least one carboxylate-substituted cyclohexene moiety; and
b) epoxidizing the intermediate obtained in step a) with an epoxidizing agent to obtain the ester comprised of at least one cyclohexene oxide moiety.

2. The method of claim 1, wherein the alcohol is a mono-alcohol.

3. The method of claim 1, wherein the alcohol is a C1 to C24 linear or branched aliphatic mono-alcohol or cycloaliphatic mono-alcohol.

4. The method of claim 1, wherein the alcohol is a polyol.

5. The method of claim 1, wherein the alcohol is a polyol selected from the group consisting of ethylene glycol; 1,2-propylene glycol; 1,3-propylene glycol; 1,2-, 1,3- and 1,4-butanediol; 1,2-, 1,3-, 1,4- and 1,5-pentanediol; 1,2-, 1,3-, 1,4-, 1,5-, and 1,6-hexanediol; 2-methyl-1,3-propanediol; neopentyl glycol; glycerol; sugars; sugar alcohols, pentaerythritol; dipentaerythritol; tripentaerythritol; trimethylolpropane; trimethylolethane; 3-methyl-1,5-pentanediol; 1,4-cyclohexanedimethanol; 1,3-cyclohexanedimethanol; oligomers of ethylene glycol, propylene glycol, and butylene glycol; bisphenols; and alkoxylated derivatives thereof.

6. The method of claim 1, wherein the alcohol contains at least one carbon-carbon double bond.

7. The method of claim 1, wherein the alcohol is selected from the group consisting of 2-ethyl hexyl alcohol, ethylene glycol, 1,2-propylene glycol, pentaerythritol, oleyl alcohol, undecelynic alcohol, benzyl alcohol, and dodecyl alcohol.

8. The method of claim 1, wherein the epoxidizing agent is a peroxy compound.

9. The method of claim 1, wherein the epoxidizing agent is a percarboxylic acid.

10. The method of claim 1, wherein the percarboxylic acid is peracetic acid.

11. The method of claim 1, wherein step a) is carried out in the presence of an acid catalyst.

12. The method of claim 11, wherein the acid catalyst has a pKa of less than −1.74.

13. The method of claim 11, wherein the acid catalyst is selected from the group consisting of sulfonic acids and sulfuric acid.

14. The method of claim 1, wherein water of reaction is removed during step a).

15. The method of claim 1, wherein the carboxylic acid-substituted cyclohexene is 3-cyclohexene-1-carboxylic acid.

16. A compound having structure (I):
Q-C(═O)O—R (I)
wherein Q is a cyclohexene oxide moiety and R is a C3-C22 alkyl or alicyclic group functionalized with one or more epoxide groups.

17. A compound having structure (II):
A-(OC(═O)Q)ₓ (II)
wherein A is an organic moiety, Q is a cyclohexene oxide moiety and x is an integer of 2 or more.
